(19) United States
(12) Patent Application Publication
(10) Pub. No.: US 2026/0196336 A1
(43) Pub. Date: Jul. 9, 2026

(54) PREDICTING PROSTATE CANCER PROGRESSION USING LONGITUDINAL BP-MRI IMAGES AND FOUNDATION MODELS

(71) Applicant: Siemens Healthineers AG, Forchheim (DE)

(72) Inventors: Bin Lou, Princeton Junction, NJ (US); Ali Kamen, Skillman, NJ (US)

(21) Appl. No.: 19/012,976

(22) Filed: Jan. 8, 2025

(51) Int. Cl. G16H 30/40; G06N 20/00; G16H 50/20; G16H 50/70

(52) U.S. Cl. CPC G16H 30/40; G06N 20/00; G16H 50/20; G16H 50/70

(57) ABSTRACT

Systems and methods for analyzing one or more anatomical objects are provided. Longitudinal medical images of one or more anatomical objects of a patient acquired over a plurality of timepoints are received. For each respective timepoint of the plurality of timepoints, features are extracted from the longitudinal medical images acquired at the respective timepoint using a machine learning based feature extractor network. The extracted features for the plurality of timepoints are weighted based on a patient atlas of the one or more anatomical objects of the patient. The one or more anatomical objects are analyzed based on the weighted extracted features using a machine learning based task network. Results of the analysis of the one or more anatomical objects are output.

### TECHNICAL FIELD

The present invention relates generally to AI/ML (artificial intelligence/machine learning) based medical imaging analysis, and in particular to predicting prostate cancer progression using longitudinal MRI (magnetic resonance imaging) images and foundation models.

### BACKGROUND

Prostate cancer is one of the most common types of cancers. Nearly half of the patients diagnosed with prostate cancer present with low-risk or favorable intermediate-risk, for which active surveillance is the recommended treatment option. Active surveillance involves regular monitoring of the prostate cancer without immediate treatment. Such monitoring typically involves follow-up examinations with PSA (prostate specific antigen) tests, mp-MRI (multi-parametric magnetic resonance imaging) imaging, and prostate biopsies. When prostate cancer lesions are in their early stages, grading MRI-detected prostate cancer legions and evaluating whether progression has occurred or will occur is a difficult and time-consuming task.

Recently, AI-based computer-aided detection systems have been proposed for the detection and assessment of prostate cancer based on mp-MRI images. However, such conventional AI-based computer-aided detection systems utilize only baseline images and follow-up images from a single follow-up active surveillance examination. Accordingly, such conventional AI-based computer-aided detection systems are unable to utilize follow-up images from prior follow-up active surveillance examinations and provide dynamic updates on progression risk.

### BRIEF SUMMARY OF THE INVENTION

In accordance with one or more embodiments, systems and methods for analyzing one or more anatomical objects are provided. Longitudinal medical images of one or more anatomical objects of a patient acquired over a plurality of timepoints are received. For each respective timepoint of the plurality of timepoints, features are extracted from the longitudinal medical images acquired at the respective timepoint using a machine learning based feature extractor network. The extracted features for the plurality of timepoints are weighted based on a patient atlas of the one or more anatomical objects of the patient. The one or more anatomical objects are analyzed based on the weighted extracted features using a machine learning based task network. Results of the analysis of the one or more anatomical objects are output.

In one embodiment, the patient atlas defines a location of the one or more anatomical objects in the longitudinal medical images.

In one embodiment, the patient atlas is generated by, for each respective timepoint of the plurality of timepoints, generating a segmentation mask of the one or more anatomical objects in the longitudinal medical images acquired at the respective timepoint. The longitudinal medical images for the plurality of timepoints are registered. The segmentation masks are combined based on the registration to generate the patient atlas.

In one embodiment, the machine learning based feature extractor network is trained by receiving training medical images. The training medical images are degraded by applying one or more transformations. Training features are extracted from the degraded training medical images using the machine learning based feature extractor network. The training medical images are reconstructed based on the extracted training features using a machine learning based decoder network. The machine learning based feature extractor network and the machine learning based decoder network are trained based on a comparison between the training medical images and reconstructed training medical images.

In one embodiment, the extracted features for the plurality of timepoints are weighted by encoding the patient atlas into a patient atlas feature vector and combining the patient atlas feature vector and the extracted features as an input token for input into the machine learning based task network.

In one embodiment, the plurality of timepoints comprises a timepoint corresponding to a baseline examination of the one or more anatomical objects, one or more timepoints corresponding to one or more follow-up examinations of the anatomical objects, and a timepoint corresponding to a current examination of the anatomical objects.

In one embodiment, the one or more anatomical objects comprise one or more prostate cancer lesions on a prostate of the patient. In one embodiment, the one or more anatomical objects are analyzed by determining at least one of a Gleason grade group score or an indication of progression of the prostate cancer lesions.

In one embodiment, the longitudinal medical images comprise medical images of an MRI (magnetic resonance imaging) sequence.

These and other advantages of the invention will be apparent to those of ordinary skill in the art by reference to the following detailed description and the accompanying drawings.

### BRIEF DESCRIPTION OF THE DRAWINGS

FIG. 1 shows a method for analyzing one or more lesions based on longitudinal medical images, in accordance with one or more embodiments;

FIG. 2 shows a workflow for analyzing one or more lesions based on longitudinal medical images, in accordance with one or more embodiments;

FIG. 3 shows a workflow for training a machine learning based feature extractor network, in accordance with one or more embodiments;

FIG. 4 shows a workflow for generating a patient atlas of one or more anatomical objects, in accordance with one or more embodiments;

FIG. 5 shows an exemplary artificial neural network that may be used to implement one or more embodiments;

FIG. 6 shows a convolutional neural network that may be used to implement one or more embodiments; and

FIG. 7 shows a high-level block diagram of a computer that may be used to implement one or more embodiments.

### DETAILED DESCRIPTION

The present invention generally relates to methods and systems for predicting prostate cancer progression using longitudinal MRI images and foundation models. Embodiments of the present invention are described herein to give a visual understanding of such methods and systems. A digital image is often composed of digital representations of one or more objects (or shapes). The digital representation of an object is often described herein in terms of identifying and manipulating the objects. Such manipulations are virtual manipulations accomplished in the memory or other circuitry/hardware of a computer system. Accordingly, is to be understood that embodiments of the present invention may be performed within a computer system using data stored within the computer system. Further, reference herein to pixels of an image may refer equally to voxels of an image and vice versa.

Embodiments described herein provide for a framework for the detection and assessment of prostate cancer lesions using longitudinal MRI images of a patient. Longitudinal features are extracted from the longitudinal MRI images using a foundation model and the extracted longitudinal features are weighted according to a patient atlas. Progression of prostate cancer lesions is then evaluated using a transformer network based on the weighted longitudinal features. Advantageously, the foundation model can accept any number of images as input, thus enabling the input of longitudinal medical MRI images from any number of timepoints. The foundation model thereby enables the generation of longitudinal features that result for improved diagnostic accuracy of prostate cancer grading and progression prediction as compared to conventional approaches.

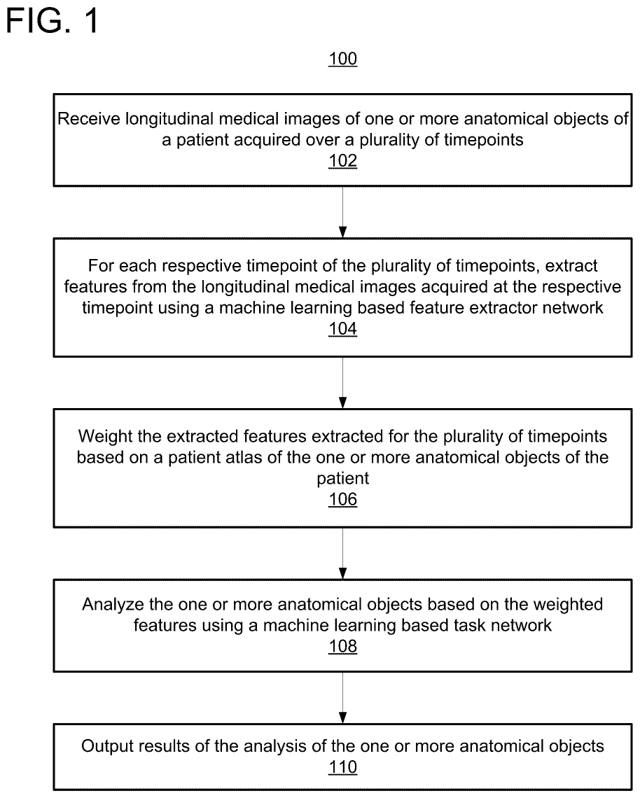

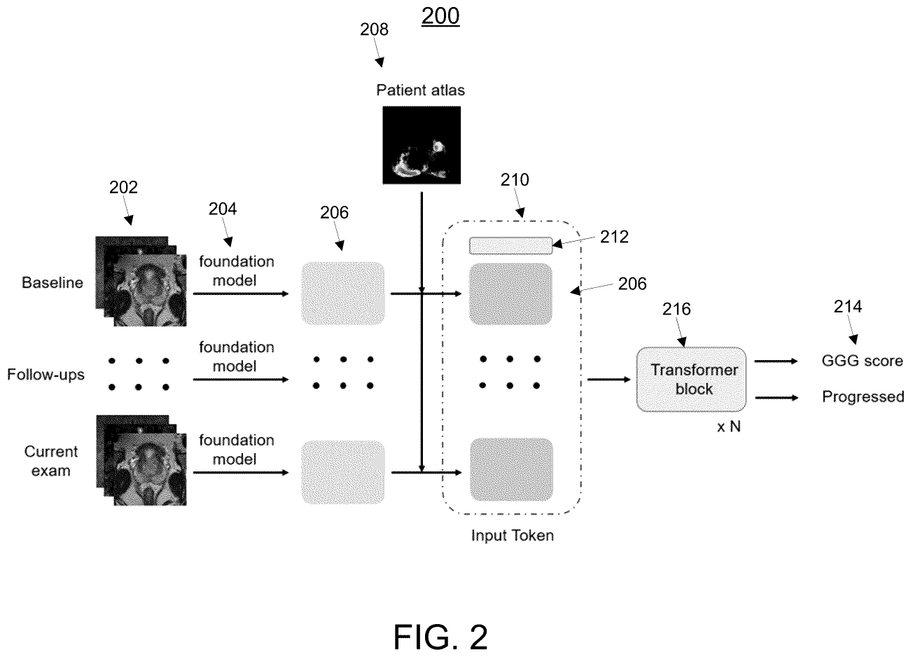

FIG. 1 shows a method 100 for analyzing one or more lesions based on longitudinal medical images, in accordance with one or more embodiments. The steps and sub-steps of method 100 may be performed by one or more suitable computing devices, such as, e.g., computer 802 of FIG. 8. FIG. 2 shows a workflow 200 for analyzing one or more lesions based on longitudinal medical images, in accordance with one or more embodiments. FIG. 1 and FIG. 2 will be described together.

At step 102 of FIG. 1, longitudinal medical images of one or more anatomical objects of a patient acquired over a plurality of timepoints are received. In one example, as shown in workflow 200 of FIG. 2, the longitudinal medical images are longitudinal medical images 202 comprising medical images acquired for a baseline examination, medical images acquired for one or more follow-up examinations, and medical images acquired for a current (e.g., most recent) examination.

In one embodiment, the one or more anatomical objects comprise lesions (e.g., prostate cancer lesions) on a prostate of the patient, as shown in longitudinal medical images 202 of FIG. 2. However, the one or more anatomical objects may comprise any other suitable anatomical object of interest, such as, e.g., tumors or other abnormalities, organs, bones, vessels, etc. The longitudinal medical images may have been acquired for monitoring progression of the one or more anatomical objects over the plurality of timepoints. For example, the longitudinal medical images may have been acquired at a first timepoint corresponding to an initial or baseline examination, one or more additional timepoints corresponding to one or more follow-up examinations, and a current timepoint corresponding to a most recent follow-up examination during active surveillance of the prostate cancer lesions.

In one embodiment, the longitudinal medical images comprise medical images of an MRI (magnetic resonance imaging) sequence, such as, e.g., T2-weighted images, ADC (apparent diffusion coefficient) maps, DWI (diffusion-weighted imaging) images, etc. However, the longitudinal medical images may comprise medical images of any other suitable domain or domains. As used herein, a domain of a medical image refers to the modality of the medical image as well as the protocol used for obtaining the medical image in that modality. The modality of the medical images may include, for example, MRI, CT (computed tomography), US (ultrasound), x-ray, SPECT (single-photon emission computed tomography), PET (positron emission tomography), or any other medical imaging modality or combinations of medical imaging modalities. The protocol used for obtaining the medical image may include, for example, acquisition sequences or techniques for acquiring a medical image, such as, e.g., T1-weighted, T2-weighted, proton density-weighted MRI images, contrast and non-contrast images, CT images captured with low kV (kilovoltage) and high kV, or low- and high-resolution medical images. Accordingly, the domains may be completely different medical imaging modalities or different image protocols within the same overall imaging modality. The first and second medical images may be 2D (two dimensional) images and/or 3D (three dimensional) volumes.

The longitudinal medical images may be received, for example, by directly receiving the longitudinal medical images from an image acquisition device (e.g., image acquisition device 814 of FIG. 8) as the images are acquired, by loading the longitudinal medical images from a storage or memory of a computer system (e.g., storage 812 or memory 810 of computer 802 of FIG. 8), or by receiving the longitudinal medical images from a remote computer system (e.g., computer 802 of FIG. 8). Such a computer system or remote computer system may comprise one or more patient databases, such as, e.g., an EHR (electronic health record), EMR (electronic medical record), PHR (personal health record), HIS (health information system), RIS (radiology information system), PACS (picture archiving and communication system), LIMS (laboratory information management system), or any other suitable database or system.

At step 104 of FIG. 1, for each respective timepoint of the plurality of timepoints, features are extracted from the longitudinal medical images acquired at the respective timepoint using a machine learning based feature extractor network. In one example, as shown in workflow 200 of FIG. 2, features 206 are respectively extracted from longitudinal medical images 202 for each of the timepoints corresponding to a baseline examination, one or more follow-up examinations, and a current examination using foundation model 204. While foundation model 204 is separately shown to illustrate the extraction of features 206 from longitudinal medical images 202 for each a baseline examination, one or more follow-up examinations, and a current examination, it should be understood that the foundation model 204 shown in workflow 200 is the same foundation model.

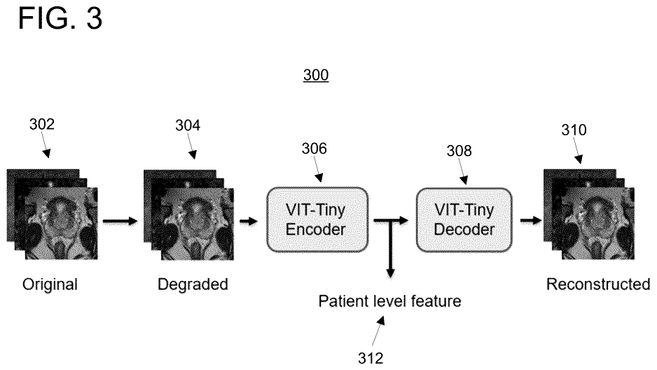

In one embodiment, the machine learning based feature extractor network is a foundation model, such as, e.g., a ViT (vision transformer). However, the machine learning based feature extractor network may be implemented according to any other suitable machine learning based architecture. The machine learning based feature extractor network receives as input longitudinal medical images for a timepoint and generates as output features for that timepoint. Accordingly, the features for the plurality of timepoints are longitudinal features. Features are low-level latent representations or embeddings of the longitudinal medical images. The machine learning based feature extractor network is trained during a prior offline or training stage, for example, according to workflow 300 of FIG. 3, as discussed in detail below. Once trained, the machine learning based feature extractor network is applied during an online or inference stage, e.g., to perform step 104 of FIG. 1.

FIG. 3 shows a workflow 300 for training a machine learning based feature extractor network, in accordance with one or more embodiments. The machine learning based feature extractor network in workflow 300 is ViT-Tiny encoder 306 of a foundation model. In one example, ViT-Tiny encoder 306 may be the machine learning based feature extractor network utilized at step 104 of FIG. 1 or foundation model 204 of FIG. 2. ViT-Tiny encoder 306 will be trained together with ViT-Tiny decoder 308 of the foundation model using self-supervised or unsupervised learning according to workflow 300 during an offline or training stage. As shown in workflow 300, original training medical images 302 are transformed into degraded images 304. In one embodiment, original training medical images 302 are medical images of an MRI sequence, but may be of any other suitable domain. Original training medical images 302 may be randomly transformed by, e.g., non-linear pixel value adjustments, inner/outer cutouts, or any other suitable transformation technique. ViT-Tiny encoder 306 receives as input degraded images 304 and generates as output patient level features 312. ViT-Tiny decoder 308 decodes patient level features 312 to generate reconstructed images 310 representing a reconstruction of original training medical images 302. ViT-Tiny encoder 306 is trained with ViT-Tiny decoder 308 by comparing original training medical images 302 with reconstructed images 310 according to a loss function, such as, e.g., MSE (mean squared errors). ViT-Tiny encoder 306 is thus trained to learn a common image representation that is both transferable and generalizable. After training, ViT-Tiny encoder 306 is applied during an inference state for feature extraction, while ViT-Tiny decoder 308 is not utilized during inference.

Returning back to FIG. 1, at step 106, the extracted features for the plurality of timepoints are weighted based on a patient atlas of the one or more anatomical objects of the patient. In one example, as shown in workflow 200 of FIG. 2, features 206 are weighted based on patient atlas 208 to provide for input token 210 of weighted features.

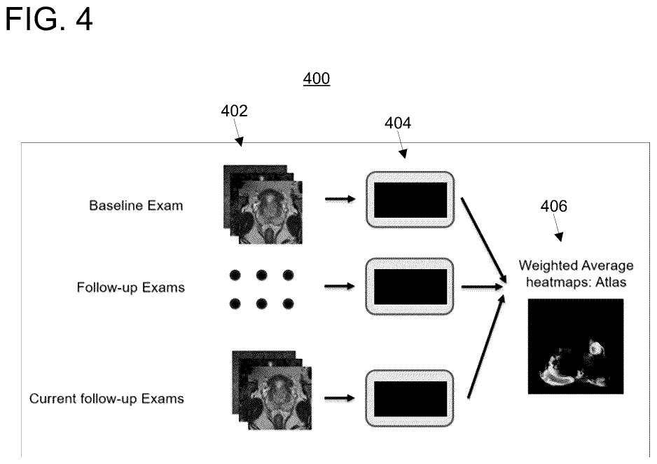

The patient atlas defines a location of the one or more anatomical objects in the longitudinal medical images. In one embodiment, the extracted features are weighted by encoding the patient atlas into a patient atlas feature vector that is in the same dimension as the extracted features. The patient atlas may be encoded, for example, using a separate machine learning based encoder network. The patient atlas feature vector and the extracted features are combined to provide for the weighted features. For example, in workflow 200 of FIG. 2, input token 210 of weighted features comprises patient atlas feature vector 212 and features 206. By weighting the extracted features based on the patient atlas, the analysis of the one or more anatomical objects (at step 108 of FIG. 1) is focused on the features corresponding to the location of the one or more anatomical objects in the longitudinal medical images. The patient atlas may be generated according to workflow 400 of FIG. 4, discussed in detail below.

FIG. 4 shows a workflow 400 for generating a patient atlas of one or more anatomical objects, in accordance with one or more embodiments. Workflow 400 may be performed to generate the patient atlas utilized at step 106 of FIG. 1. As shown in workflow 400, training longitudinal medical images 502 are received and a segmentation mask (or heatmap) 404 of the one or more anatomical objects in training longitudinal medical images 402 is generated for each of the plurality of timepoints. The segmentation masks 404 may be generated using a machine learning based segmentation network. The machine learning based segmentation network receives as input training longitudinal medical images 402 for a particular timepoint and generates a segmentation mask 404 of the one or more anatomical objects as output. The segmentation mask 404 provides for a pixel-wise location of the one or more anatomical objects in the training longitudinal medical images 402 for that particular timepoint. The intensity value of each pixel of the segmentation mask 404 indicates the probability of the presence of the one or more anatomical objects at that pixel of the training longitudinal medical images 402 for the particular timepoint and reflects the associated malignancy risk. Segmentation masks 404 may be generated according to any other suitable (e.g., well-known) approach.

The training longitudinal medical images 402 for the plurality of timepoints are registered, e.g., using any suitable (e.g., well-known) registration technique. Segmentation masks 404 for the plurality of timepoints are combined to generate patient atlas 406 based on the registration. In one embodiment, segmentation masks 404 are combined by averaging the intensity values of pixels of the segmentation masks 404 corresponding to a same location, as determined based on the registration. The averaging may be a weighted averaging with pixel intensity values of segmentation masks 404 extracted from more recent training longitudinal medical images 402 given more weight.

Returning to FIG. 1, at step 108, the one or more anatomical objects are analyzed based on the weighted features using a machine learning based task network. In one example, as shown in workflow 200 of FIG. 2, analysis of the lesions is performed using transformer block 216 based on input token 210 to determine analysis results 214 comprising GGG (Gleason grade group) score and an indication that the lesions have progressed.

In one embodiment, the machine learning based task network comprises a transformer network. However, the machine learning based task network may be implemented according to any other suitable machine learning based architecture. The machine learning based task network receives as input the weighted features (i.e., the patient atlas feature vector and the extracted features) and generates as output one or more results of the analysis. In one example, the analysis of the one or more anatomical objects may comprise a determine of a GGG (Gleason grade group) score and/or an indication of progression of the one or more anatomical objects (e.g., progressed or has not progressed). However, the analysis of the one or more anatomical objects may comprise any other suitable medical imaging analysis task.

At step 110 of FIG. 1, results of the analysis of the one or more anatomical objects are output. For example, the results of the analysis of the one or more anatomical objects can be output by displaying the results on a display device of a computer system (e.g., I/O 808 of computer 802 of FIG. 8), storing the results on a memory or storage of a computer system (e.g., memory 810 or storage 812 of computer 802 of FIG. 8), or by transmitting the results to a remote computer system (e.g., computer 802 of FIG. 8).

Advantageously, embodiments described herein enable the efficient integration of MRI images from any number of prior examinations and enhancing the accuracy of prostate cancer grading and progression prediction. Embodiments described herein utilized self-supervised learning techniques to develop the machine learning based encoder network, which can leverage extensive induvial single-time prostate MRI images to improve the encoder network's effectiveness in addressing longitudinal prostate cancer active surveillance challenges. The machine learning based encoder network in accordance with embodiments described herein integrates longitudinal features, which not only improves diagnostic accuracy but also supports personalized treatment planning by considering both longitudinal data and specific characteristics of individual lesions. Embodiments described herein can be generalized for analyzing prostate cancer, as well as any other disease the utilized follow-up examination of a patient's condition without immediate treatment.

Embodiments described herein are described with respect to the claimed systems as well as with respect to the claimed methods. Features, advantages or alternative embodiments herein can be assigned to the other claimed objects and vice versa. In other words, claims and embodiments for the systems can be improved with features described or claimed in the context of the respective methods. In this case, the functional features of the method are implemented by physical units of the system.

Furthermore, certain embodiments described herein are described with respect to methods and systems utilizing trained machine learning models, as well as with respect to methods and systems for providing trained machine learning models. Features, advantages or alternative embodiments herein can be assigned to the other claimed objects and vice versa. In other words, claims and embodiments for providing trained machine learning models can be improved with features described or claimed in the context of utilizing trained machine learning models, and vice versa. In particular, datasets used in the methods and systems for utilizing trained machine learning models can have the same properties and features as the corresponding datasets used in the methods and systems for providing trained machine learning models, and the trained machine learning models provided by the respective methods and systems can be used in the methods and systems for utilizing the trained machine learning models.

In general, a trained machine learning model mimics cognitive functions that humans associate with other human minds. In particular, by training based on training data the machine learning model is able to adapt to new circumstances and to detect and extrapolate patterns. Another term for “trained machine learning model” is “trained function.”

In general, parameters of a machine learning model can be adapted by means of training. In particular, supervised training, semi-supervised training, unsupervised training, reinforcement learning and/or active learning can be used. Furthermore, representation learning (an alternative term is “feature learning”) can be used. In particular, the parameters of the machine learning models can be adapted iteratively by several steps of training. In particular, within the training a certain cost function can be minimized. In particular, within the training of a neural network the backpropagation algorithm can be used.

In particular, a machine learning model, such as, e.g., the machine learning based feature extractor network utilized at step 104 and the machine learning based task network utilized at step 108 of FIG. 1, foundation model 204 and transformer block 216 of FIG. 2, and ViT-Tiny encoder 306 and ViT-Tiny decoder 308 of FIG. 3, can comprise, for example, a neural network, a support vector machine, a decision tree and/or a Bayesian network, and/or the machine learning model can be based on, for example, k-means clustering, Q-learning, genetic algorithms and/or association rules. In particular, a neural network can be, e.g., a deep neural network, a convolutional neural network or a convolutional deep neural network. Furthermore, a neural network can be, e.g., an adversarial network, a deep adversarial network and/or a generative adversarial network.

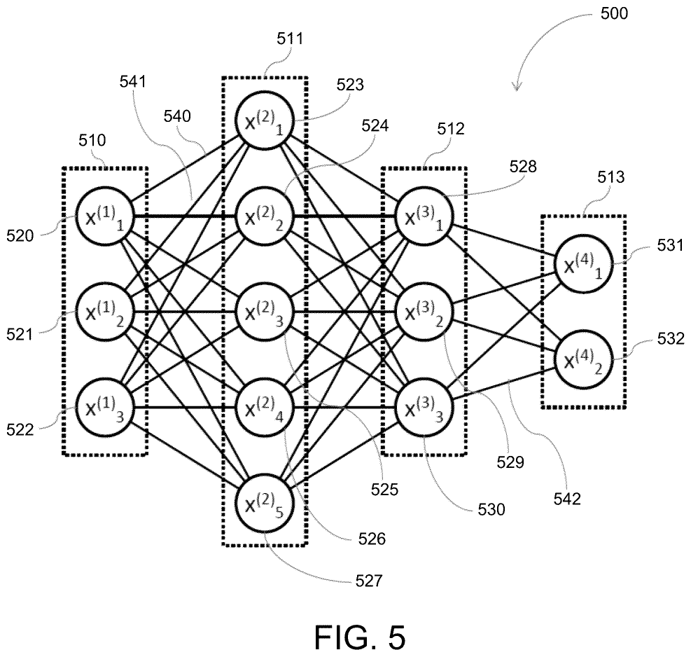

FIG. 5 shows an embodiment of an artificial neural network 500 that may be used to implement one or more machine learning models described herein. AIternative terms for “artificial neural network” are “neural network”, “artificial neural net” or “neural net”.

The artificial neural network 500 comprises nodes 520, . . . , 532 and edges 540, . . . , 542, wherein each edge 540, . . . , 542 is a directed connection from a first node 520, . . . , 532 to a second node 520, . . . , 532. In general, the first node 520, . . . , 532 and the second node 520, . . . , 532 are different nodes 520, . . . , 532, it is also possible that the first node 520, . . . , 532 and the second node 520, . . . , 532 are identical. For example, in FIG. 5 the edge 540 is a directed connection from the node 520 to the node 523, and the edge 542 is a directed connection from the node 530 to the node 532. An edge 540, . . . , 542 from a first node 520, . . . , 532 to a second node 520, . . . , 532 is also denoted as “ingoing edge” for the second node 520, . . . , 532 and as “outgoing edge” for the first node 520, . . . , 532.

In this embodiment, the nodes 520, . . . , 532 of the artificial neural network 500 can be arranged in layers 510, . . . , 513, wherein the layers can comprise an intrinsic order introduced by the edges 540, . . . , 542 between the nodes 520, . . . , 532. In particular, edges 540, . . . , 542 can exist only between neighboring layers of nodes. In the displayed embodiment, there is an input layer 510 comprising only nodes 520, . . . , 522 without an incoming edge, an output layer 513 comprising only nodes 531, 532 without outgoing edges, and hidden layers 511, 512 in-between the input layer 510 and the output layer 513. In general, the number of hidden layers 511, 512 can be chosen arbitrarily. The number of nodes 520, . . . , 522 within the input layer 510 usually relates to the number of input values of the neural network, and the number of nodes 531, 532 within the output layer 513 usually relates to the number of output values of the neural network.

In particular, a (real) number can be assigned as a value to every node 520, . . . , 532 of the neural network 500. Here, x⁽ⁿ⁾ᵢ denotes the value of the i-th node 520, . . . , 532 of the n-th layer 510, . . . , 513. The values of the nodes 520, . . . , 522 of the input layer 510 are equivalent to the input values of the neural network 500, the values of the nodes 531, 532 of the output layer 513 are equivalent to the output value of the neural network 500. Furthermore, each edge 540, . . . , 542 can comprise a weight being a real number, in particular, the weight is a real number within the interval [−1, 1] or within the interval [0, 1]. Here, w^{(m,n)}_{i,j} denotes the weight of the edge between the i-th node 520, . . . , 532 of the m-th layer 510, . . . , 513 and the j-th node 520, . . . , 532 of the n-th layer 510, . . . , 513. Furthermore, the abbreviation w⁽ⁿ⁾_{i,j} is defined for the weight w^{(n,n+1)}_{i,j}.

In particular, to calculate the output values of the neural network 500, the input values are propagated through the neural network. In particular, the values of the nodes 520, . . . , 532 of the (n+1)-th layer 510, . . . , 513 can be calculated based on the values of the nodes 520, . . . , 532 of the n-th layer 510, . . . , 513 by

$${x}^{{( n + 1 )}_{j}} = f ( {\sum }_{i} {x}^{{( n )}_{i}} \cdot {w}^{{( n )}_{i , j}} ) .$$

Herein, the function f is a transfer function (another term is “activation function”). Known transfer functions are step functions, sigmoid function (e.g., the logistic function, the generalized logistic function, the hyperbolic tangent, the Arctangent function, the error function, the smoothstep function) or rectifier functions. The transfer function is mainly used for normalization purposes.

In particular, the values are propagated layer-wise through the neural network, wherein values of the input layer 510 are given by the input of the neural network 500, wherein values of the first hid-den layer 511 can be calculated based on the values of the input layer 510 of the neural network, wherein values of the second hidden layer 512 can be calculated based in the values of the first hidden layer 511, etc.

In order to set the values w^{(m,n)}_{i,j} for the edges, the neural network 500 has to be trained using training data. In particular, training data comprises training input data and training output data (denoted as tᵢ). For a training step, the neural network 500 is applied to the training input data to generate calculated output data. In particular, the training data and the calculated output data comprise a number of values, said number being equal with the number of nodes of the output layer.

In particular, a comparison between the calculated output data and the training data is used to recursively adapt the weights within the neural network 500 (backpropagation algorithm). In particular, the weights are changed according to

$${w}^{{′ ( n )}_{i , j}} = {w}^{{( n )}_{i , j}} - y \cdot {δ}^{{( n )}_{j}} \cdot {x}^{{( n )}_{i}}$$

wherein γ is a learning rate, and the numbers δ⁽ⁿ⁾ᵢ can be recursively calculated as

$${δ}^{{( n )}_{j}} = ( {\sum }_{k} {δ}^{{( n + 1 )}_{k}} \cdot {w}^{{( n + 1 )}_{j , k}} ) \cdot {f}^{′} ( {\sum }_{i} {x}^{{( n )}_{i}} \cdot {w}^{{( n )}_{i , j}} )$$

based on δ⁽ⁿ⁺¹⁾ⱼ, if the (n+1)-th layer is not the output layer, and

$${δ}^{{( n )}_{j}} = ( {x}^{{( n + 1 )}_{j}} - {t}^{{( n + 1 )}_{j}} ) \cdot {f}^{′} ( {x}^{{( n )}_{i}} \cdot {w}^{{( n )}_{i , j}} )$$

if the (n+1)-th layer is the output layer 513, wherein f′ is the first derivative of the activation function, and t⁽ⁿ⁺¹⁾ⱼ is the comparison training value for the j-th node of the output layer 513.

A convolutional neural network is a neural network that uses a convolution operation instead of general matrix multiplication in at least one of its layers (so-called “convolutional layer”). In particular, a convolutional layer performs a dot product of one or more convolution kernels with the convolutional layer's input data/image, wherein the entries of the one or more convolution kernels are the parameters or weights that are adapted by training. In particular, one can use the Frobenius inner product and the ReLU activation function. A convolutional neural network can comprise additional layers, e.g., pooling layers, fully connected layers, and normalization layers.

By using convolutional neural networks input images can be processed in a very efficient way, because a convolution operation based on different kernels can extract various image features, so that by adapting the weights of the convolution kernel the relevant image features can be found during training. Furthermore, based on the weight-sharing in the convolutional kernels less parameters need to be trained, which prevents overfitting in the training phase and allows to have faster training or more layers in the network, improving the performance of the network.

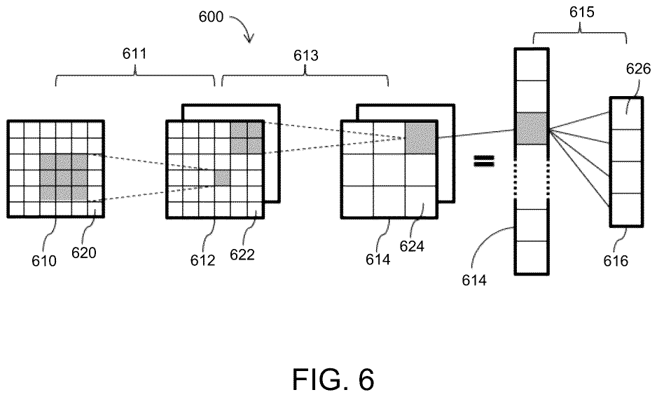

FIG. 6 shows an embodiment of a convolutional neural network 600 that may be used to implement one or more machine learning models described herein. In the displayed embodiment, the convolutional neural network 600 comprises an input node layer 610, a convolutional layer 611, a pooling layer 613, a fully connected layer 614 and an output node layer 616, as well as hidden node layers 612, 614. AIternatively, the convolutional neural network 600 can comprise several convolutional layers 611, several pooling layers 613 and several fully connected layers 615, as well as other types of layers. The order of the layers can be chosen arbitrarily, usually fully connected layers 615 are used as the last layers before the output layer 616.

In particular, within a convolutional neural network 600 nodes 620, 622, 624 of a node layer 610, 612, 614 can be considered to be arranged as a d-dimensional matrix or as a d-dimensional image. In particular, in the two-dimensional case the value of the node 620, 622, 624 indexed with i and j in the n-th node layer 610, 612, 614 can be denoted as x(n)[i,j]. However, the arrangement of the nodes 620, 622, 624 of one node layer 610, 612, 614 does not have an effect on the calculations executed within the convolutional neural network 600 as such, since these are given solely by the structure and the weights of the edges.

A convolutional layer 611 is a connection layer between an anterior node layer 610 (with node values x(n−1)) and a posterior node layer 612 (with node values x(n)). In particular, a convolutional layer 611 is characterized by the structure and the weights of the incoming edges forming a convolution operation based on a certain number of kernels. In particular, the structure and the weights of the edges of the convolutional layer 611 are chosen such that the values x(n) of the nodes 622 of the posterior node layer 612 are calculated as a convolution x(n)=K*x(n−1) based on the values x(n−1) of the nodes 620 anterior node layer 610, where the convolution * is defined in the two-dimensional case as

$${x}_{k}^{( n )} [ i , j ] = ( K * {x}^{( n - 1 )} ) [ i , j ] = {\sum }_{{i}^{′}} {\sum }_{{j}^{′}} K [ {i}^{′} , {j}^{′} ] \cdot {x}^{( n - 1 )} [ i - {i}^{′} , j - {j}^{′} ] .$$

Here the kernel K is a d-dimensional matrix (in this embodiment, a two-dimensional matrix), which is usually small compared to the number of nodes 620, 622 (e.g., a 3×3 matrix, or a 5×5 matrix). In particular, this implies that the weights of the edges in the convolution layer 611 are not independent, but chosen such that they produce said convolution equation. In particular, for a kernel being a 3×3 matrix, there are only 9 independent weights (each entry of the kernel matrix corresponding to one independent weight), irrespectively of the number of nodes 620, 622 in the anterior node layer 610 and the posterior node layer 612.

In general, convolutional neural networks 600 use node layers 610, 612, 614 with a plurality of channels, in particular, due to the use of a plurality of kernels in convolutional layers 611. In those cases, the node layers can be considered as (d+1)-dimensional matrices (the first dimension indexing the channels). The action of a convolutional layer 611 is then a two-dimensional example defined as

$${x}^{{( n )}_{b}} [ i , j ] = {\sum }_{a} {K}_{a , b} * {x}^{{( n - 1 )}_{a}} [ i , j ] = {\sum }_{a} {\sum }_{{i}^{′}} {\sum }_{{j}^{′}} {K}_{a , b} [ {i}^{′} , {j}^{′} ] \cdot {x}^{{( n - 1 )}_{a}} [ i - {i}^{′} , j - {j}^{′} ]$$

where x⁽ⁿ⁻¹⁾^{ₐ} corresponds to the a-th channel of the anterior node layer 610, x⁽ⁿ⁾^{_{b}} corresponds to the b-th channel of the posterior node layer 612 and K_{a,b} corresponds to one of the kernels. If a convolutional layer 611 acts on an anterior node layer 610 with A channels and outputs a posterior node layer 612 with B channels, there are A·B independent d-dimensional kernels K_{a,b}.

In general, in convolutional neural networks 600 activation functions are used. In this embodiment ReLU (acronym for “Rectified Linear Units”) is used, with R(z)=max(0, z), so that the action of the convolutional layer 611 in the two-dimensional example is

$$\begin{matrix}{x}^{{( n )}_{b}} [ i , j ] = R ( {\sum }_{a} ( {K}_{a , b} * {x}^{{( n - 1 )}_{a}} ) [ i , j ] ) \\ = R ( {\sum }_{a} {\sum }_{{i}^{′}} {\sum }_{{j}^{′}} {K}_{a , b} [ {i}^{′} , {j}^{′} ] \cdot {x}^{{( n - 1 )}_{a}} [ i - {i}^{′} , j - {j}^{′} ] )\end{matrix}$$

It is also possible to use other activation functions, e.g., ELU (acronym for “Exponential Linear Unit”), LeakyReLU, Sigmoid, Tanh or Softmax.

In the displayed embodiment, the input layer 610 comprises 36 nodes 620, arranged as a two-dimensional 6×6 matrix. The first hidden node layer 612 comprises 72 nodes 622, arranged as two two-dimensional 6×6 matrices, each of the two matrices being the result of a convolution of the values of the input layer with a 3×3 kernel within the convolutional layer 611. Equivalently, the nodes 622 of the first hidden node layer 612 can be interpreted as arranged as a three-dimensional 2×6×6 matrix, wherein the first dimension correspond to the channel dimension.

The advantage of using convolutional layers 611 is that spatially local correlation of the input data can exploited by enforcing a local connectivity pattern between nodes of adjacent layers, in particular by each node being connected to only a small region of the nodes of the preceding layer.

A pooling layer 613 is a connection layer between an anterior node layer 612 (with node values x(n−1)) and a posterior node layer 614 (with node values x(n)). In particular, a pooling layer 613 can be characterized by the structure and the weights of the edges and the activation function forming a pooling operation based on a non-linear pooling function f. For example, in the two-dimensional case the values x(n) of the nodes 624 of the posterior node layer 614 can be calculated based on the values x(n−1) of the nodes 622 of the anterior node layer 612 as

$${x}^{{( n )}_{b}} [ i , j ] = f \{ {x}^{( n - 1 )} [ {id}_{1} , {jd}_{2} ] , … , {x}^{{( n - 1 )}_{b}} [ ( i + 1 ) {d}_{1} - 1 , ( j + 1 ) {d}_{2} - 1 ] )$$

In other words, by using a pooling layer 613 the number of nodes 622, 624 can be reduced, by re-placing a number d1·d2 of neighboring nodes 622 in the anterior node layer 612 with a single node 622 in the posterior node layer 614 being calculated as a function of the values of said number of neighboring nodes. In particular, the pooling function f can be the max-function, the average or the L2-Norm. In particular, for a pooling layer 613 the weights of the incoming edges are fixed and are not modified by training.

The advantage of using a pooling layer 613 is that the number of nodes 622, 624 and the number of parameters is reduced. This leads to the amount of computation in the network being reduced and to a control of overfitting.

In the displayed embodiment, the pooling layer 613 is a max-pooling layer, replacing four neighboring nodes with only one node, the value being the maximum of the values of the four neighboring nodes. The max-pooling is applied to each d-dimensional matrix of the previous layer; in this embodiment, the max-pooling is applied to each of the two two-dimensional matrices, reducing the number of nodes from 72 to 18.

In general, the last layers of a convolutional neural network 600 are fully connected layers 615. A fully connected layer 615 is a connection layer between an anterior node layer 614 and a posterior node layer 616. A fully connected layer 613 can be characterized by the fact that a majority, in particular, all edges between nodes 614 of the anterior node layer 614 and the nodes 616 of the posterior node layer are present, and wherein the weight of each of these edges can be adjusted individually.

In this embodiment, the nodes 624 of the anterior node layer 614 of the fully connected layer 615 are displayed both as two-dimensional matrices, and additionally as non-related nodes (indicated as a line of nodes, wherein the number of nodes was reduced for a better presentability). This operation is also denoted as “flattening”. In this embodiment, the number of nodes 626 in the posterior node layer 616 of the fully connected layer 615 smaller than the number of nodes 624 in the anterior node layer 614. AIternatively, the number of nodes 626 can be equal or larger.

Furthermore, in this embodiment the Softmax activation function is used within the fully connected layer 615. By applying the Softmax function, the sum the values of all nodes 626 of the output layer 616 is 1, and all values of all nodes 626 of the output layer 616 are real numbers between 0 and 1. In particular, if using the convolutional neural network 600 for categorizing input data, the values of the output layer 616 can be interpreted as the probability of the input data falling into one of the different categories.

In particular, convolutional neural networks 600 can be trained based on the backpropagation algorithm. For preventing overfitting, methods of regularization can be used, e.g., dropout of nodes 620, . . . , 624, stochastic pooling, use of artificial data, weight decay based on the L1 or the L2 norm, or max norm constraints.

According to an aspect, the machine learning model may comprise one or more residual networks (ResNet). In particular, a ResNet is an artificial neural network comprising at least one jump or skip connection used to jump over at least one layer of the artificial neural network. In particular, a ResNet may be a convolutional neural network comprising one or more skip connections respectively skipping one or more convolutional layers. According to some examples, the ResNets may be represented as m-layer ResNets, where m is the number of layers in the corresponding architecture and, according to some examples, may take values of 34, 50, 101, or 152. According to some examples, such an m-layer ResNet may respectively comprise (m−2)/2 skip connections.

A skip connection may be seen as a bypass which directly feeds the output of one preceding layer over one or more bypassed layers to a layer succeeding the one or more bypassed layers. Instead of having to directly fit a desired mapping, the bypassed layers would then have to fit a residual mapping “balancing” the directly fed output.

Fitting the residual mapping is computationally easier to optimize than the directed mapping. What is more, this alleviates the problem of vanishing/exploding gradients during optimization upon training the machine learning models: if a bypassed layer runs into such problems, its contribution may be skipped by regularization of the directly fed output. Using ResNets thus brings about the advantage that much deeper networks may be trained.

Systems, apparatuses, and methods described herein may be implemented using digital circuitry, or using one or more computers using well-known computer processors, memory units, storage devices, computer software, and other components. Typically, a computer includes a processor for executing instructions and one or more memories for storing instructions and data. A computer may also include, or be coupled to, one or more mass storage devices, such as one or more magnetic disks, internal hard disks and removable disks, magneto-optical disks, optical disks, etc.

Systems, apparatuses, and methods described herein may be implemented using computers operating in a client-server relationship. Typically, in such a system, the client computers are located remotely from the server computer and interact via a network. The client-server relationship may be defined and controlled by computer programs running on the respective client and server computers.

Systems, apparatuses, and methods described herein may be implemented within a network-based cloud computing system. In such a network-based cloud computing system, a server or another processor that is connected to a network communicates with one or more client computers via a network. A client computer may communicate with the server via a network browser application residing and operating on the client computer, for example. A client computer may store data on the server and access the data via the network. A client computer may transmit requests for data, or requests for online services, to the server via the network. The server may perform requested services and provide data to the client computer(s). The server may also transmit data adapted to cause a client computer to perform a specified function, e.g., to perform a calculation, to display specified data on a screen, etc. For example, the server may transmit a request adapted to cause a client computer to perform one or more of the steps or functions of the methods and workflows described herein, including one or more of the steps or functions of FIGS. 1-4. Certain steps or functions of the methods and workflows described herein, including one or more of the steps or functions of FIGS. 1-4, may be performed by a server or by another processor in a network-based cloud-computing system. Certain steps or functions of the methods and workflows described herein, including one or more of the steps of FIGS. 1-4, may be performed by a client computer in a network-based cloud computing system. The steps or functions of the methods and workflows described herein, including one or more of the steps of FIGS. 1-4, may be performed by a server and/or by a client computer in a network-based cloud computing system, in any combination.

Systems, apparatuses, and methods described herein may be implemented using a computer program product tangibly embodied in an information carrier, e.g., in a non-transitory machine-readable storage device, for execution by a programmable processor; and the method and workflow steps described herein, including one or more of the steps or functions of FIGS. 1-4, may be implemented using one or more computer programs that are executable by such a processor. A computer program is a set of computer program instructions that can be used, directly or indirectly, in a computer to perform a certain activity or bring about a certain result. A computer program can be written in any form of programming language, including compiled or interpreted languages, and it can be deployed in any form, including as a stand-alone program or as a module, component, subroutine, or other unit suitable for use in a computing environment.

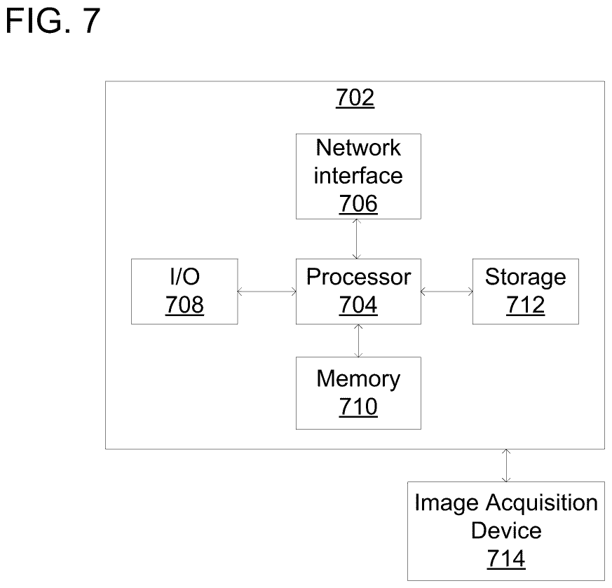

A high-level block diagram of an example computer 702 that may be used to implement systems, apparatuses, and methods described herein is depicted in FIG. 7. Computer 702 includes a processor 704 operatively coupled to a data storage device 712 and a memory 710. Processor 704 controls the overall operation of computer 702 by executing computer program instructions that define such operations. The computer program instructions may be stored in data storage device 712, or other computer readable medium, and loaded into memory 710 when execution of the computer program instructions is desired. Thus, the method and workflow steps or functions of FIGS. 1-4 can be defined by the computer program instructions stored in memory 710 and/or data storage device 712 and controlled by processor 704 executing the computer program instructions. For example, the computer program instructions can be implemented as computer executable code programmed by one skilled in the art to perform the method and workflow steps or functions of FIGS. 1-4. Accordingly, by executing the computer program instructions, the processor 704 executes the method and workflow steps or functions of FIGS. 1-4. Computer 702 may also include one or more network interfaces 706 for communicating with other devices via a network. Computer 702 may also include one or more input/output devices 708 that enable user interaction with computer 702 (e.g., display, keyboard, mouse, speakers, buttons, etc.).

Processor 704 may include both general and special purpose microprocessors, and may be the sole processor or one of multiple processors of computer 702. Processor 704 may include one or more central processing units (CPUs), for example. Processor 704, data storage device 712, and/or memory 710 may include, be supplemented by, or incorporated in, one or more application-specific integrated circuits (ASICs) and/or one or more field programmable gate arrays (FPGAs).

Data storage device 712 and memory 710 each include a tangible non-transitory computer readable storage medium. Data storage device 712, and memory 710, may each include high-speed random access memory, such as dynamic random access memory (DRAM), static random access memory (SRAM), double data rate synchronous dynamic random access memory (DDR RAM), or other random access solid state memory devices, and may include non-volatile memory, such as one or more magnetic disk storage devices such as internal hard disks and removable disks, magneto-optical disk storage devices, optical disk storage devices, flash memory devices, semiconductor memory devices, such as erasable programmable read-only memory (EPROM), electrically erasable programmable read-only memory (EEPROM), compact disc read-only memory (CD-ROM), digital versatile disc read-only memory (DVD-ROM) disks, or other non-volatile solid state storage devices.

Input/output devices 708 may include peripherals, such as a printer, scanner, display screen, etc. For example, input/output devices 708 may include a display device such as a cathode ray tube (CRT) or liquid crystal display (LCD) monitor for displaying information to the user, a keyboard, and a pointing device such as a mouse or a trackball by which the user can provide input to computer 702.

An image acquisition device 714 can be connected to the computer 702 to input image data (e.g., medical images) to the computer 702. It is possible to implement the image acquisition device 714 and the computer 702 as one device. It is also possible that the image acquisition device 714 and the computer 702 communicate wirelessly through a network. In a possible embodiment, the computer 702 can be located remotely with respect to the image acquisition device 714.

Any or all of the systems, apparatuses, and methods discussed herein may be implemented using one or more computers such as computer 702.

One skilled in the art will recognize that an implementation of an actual computer or computer system may have other structures and may contain other components as well, and that FIG. 7 is a high level representation of some of the components of such a computer for illustrative purposes.

Independent of the grammatical term usage, individuals with male, female or other gender identities are included within the term.

The foregoing Detailed Description is to be understood as being in every respect illustrative and exemplary, but not restrictive, and the scope of the invention disclosed herein is not to be determined from the Detailed Description, but rather from the claims as interpreted according to the full breadth permitted by the patent laws. It is to be understood that the embodiments shown and described herein are only illustrative of the principles of the present invention and that various modifications may be implemented by those skilled in the art without departing from the scope and spirit of the invention. Those skilled in the art could implement various other feature combinations without departing from the scope and spirit of the invention.

The following is a list of non-limiting illustrative embodiments disclosed herein:

Illustrative embodiment 1. A computer-implemented method comprising: receiving longitudinal medical images of one or more anatomical objects of a patient acquired over a plurality of timepoints; for each respective timepoint of the plurality of timepoints, extracting features from the longitudinal medical images acquired at the respective timepoint using a machine learning based feature extractor network; weighting the extracted features for the plurality of timepoints based on a patient atlas of the one or more anatomical objects of the patient; analyzing the one or more anatomical objects based on the weighted extracted features using a machine learning based task network; and outputting results of the analysis of the one or more anatomical objects.

Illustrative embodiment 2. The computer-implemented method of illustrative embodiment 1, wherein the patient atlas defines a location of the one or more anatomical objects in the longitudinal medical images.

Illustrative embodiment 3. The computer-implemented method of any one of illustrative embodiments 1-2, further comprising generating the patient atlas by: for each respective timepoint of the plurality of timepoints, generating a segmentation mask of the one or more anatomical objects in the longitudinal medical images acquired at the respective timepoint; registering the longitudinal medical images for the plurality of timepoints; and combining the segmentation masks based on the registration to generate the patient atlas.

Illustrative embodiment 4. The computer-implemented method of any one of illustrative embodiments 1-3, wherein the machine learning based feature extractor network is trained by: receiving training medical images; degrading the training medical images by applying one or more transformations; extracting training features from the degraded training medical images using the machine learning based feature extractor network; reconstructing the training medical images based on the extracted training features using a machine learning based decoder network; and training the machine learning based feature extractor network and the machine learning based decoder network based on a comparison between the training medical images and reconstructed training medical images.

Illustrative embodiment 5. The computer-implemented method of any one of illustrative embodiments 1-4, wherein weighting the extracted features for the plurality of timepoints based on a patient atlas of the one or more anatomical objects of the patient comprises: encoding the patient atlas into a patient atlas feature vector; and combining the patient atlas feature vector and the extracted features as an input token for input into the machine learning based task network.

Illustrative embodiment 6. The computer-implemented method of any one of illustrative embodiments 1-5, wherein the plurality of timepoints comprises a timepoint corresponding to a baseline examination of the one or more anatomical objects, one or more timepoints corresponding to one or more follow-up examinations of the anatomical objects, and a timepoint corresponding to a current examination of the anatomical objects.

Illustrative embodiment 7. The computer-implemented method of any one of illustrative embodiments 1-6, wherein the one or more anatomical objects comprise one or more prostate cancer lesions on a prostate of the patient.

Illustrative embodiment 8. The computer-implemented method of illustrative embodiment 7, wherein analyzing the one or more anatomical objects based on the weighted extracted features using a machine learning based task network comprises: determining at least one of a Gleason grade group score or an indication of progression of the prostate cancer lesions.

Illustrative embodiment 9. The computer-implemented method of any one of illustrative embodiments 1-8, wherein the longitudinal medical images comprise medical images of an MRI (magnetic resonance imaging) sequence.

Illustrative embodiment 10. An apparatus comprising: means for receiving longitudinal medical images of one or more anatomical objects of a patient acquired over a plurality of timepoints; for each respective timepoint of the plurality of timepoints, means for extracting features from the longitudinal medical images acquired at the respective timepoint using a machine learning based feature extractor network; means for weighting the extracted features for the plurality of timepoints based on a patient atlas of the one or more anatomical objects of the patient; means for analyzing the one or more anatomical objects based on the weighted extracted features using a machine learning based task network; and means for outputting results of the analysis of the one or more anatomical objects.

Illustrative embodiment 11. The apparatus of illustrative embodiment 10, wherein the patient atlas defines a location of the one or more anatomical objects in the longitudinal medical images.

Illustrative embodiment 12. The apparatus of any one of illustrative embodiments 10-11, further comprising means for generating the patient atlas by: for each respective timepoint of the plurality of timepoints, generating a segmentation mask of the one or more anatomical objects in the longitudinal medical images acquired at the respective timepoint; registering the longitudinal medical images for the plurality of timepoints; and combining the segmentation masks based on the registration to generate the patient atlas.

Illustrative embodiment 13. The apparatus of any one of illustrative embodiments 10-12, wherein the machine learning based feature extractor network is trained by: means for receiving training medical images; means for degrading the training medical images by applying one or more transformations; means for extracting training features from the degraded training medical images using the machine learning based feature extractor network; means for reconstructing the training medical images based on the extracted training features using a machine learning based decoder network; and means for training the machine learning based feature extractor network and the machine learning based decoder network based on a comparison between the training medical images and reconstructed training medical images.

Illustrative embodiment 14. The apparatus of any one of illustrative embodiments 10-13, wherein the means for weighting the extracted features for the plurality of timepoints based on a patient atlas of the one or more anatomical objects of the patient comprises: means for encoding the patient atlas into a patient atlas feature vector; and means for combining the patient atlas feature vector and the extracted features as an input token for input into the machine learning based task network.

Illustrative embodiment 15. A non-transitory computer-readable storage medium comprising instructions which, when executed by a computer, cause the computer to carry out operations comprising: receiving longitudinal medical images of one or more anatomical objects of a patient acquired over a plurality of timepoints; for each respective timepoint of the plurality of timepoints, extracting features from the longitudinal medical images acquired at the respective timepoint using a machine learning based feature extractor network; weighting the extracted features for the plurality of timepoints based on a patient atlas of the one or more anatomical objects of the patient; analyzing the one or more anatomical objects based on the weighted extracted features using a machine learning based task network; and outputting results of the analysis of the one or more anatomical objects.

Illustrative embodiment 16. The non-transitory computer-readable storage medium of illustrative embodiment 15, wherein the patient atlas defines a location of the one or more anatomical objects in the longitudinal medical images.

Illustrative embodiment 17. The non-transitory computer-readable storage medium of any one of illustrative embodiments 15-16, wherein the plurality of timepoints comprises a timepoint corresponding to a baseline examination of the one or more anatomical objects, one or more timepoints corresponding to one or more follow-up examinations of the anatomical objects, and a timepoint corresponding to a current examination of the anatomical objects.

Illustrative embodiment 18. The non-transitory computer-readable storage medium of any one of illustrative embodiments 15-17, wherein the one or more anatomical objects comprise one or more prostate cancer lesions on a prostate of the patient.

Illustrative embodiment 19. The non-transitory computer-readable storage medium of any one of illustrative embodiments 15-18, wherein analyzing the one or more anatomical objects based on the weighted extracted features using a machine learning based task network comprises: determining at least one of a Gleason grade group score or an indication of progression of the prostate cancer lesions.

Illustrative embodiment 20. The non-transitory computer-readable storage medium of any one of illustrative embodiments 15-19, wherein the longitudinal medical images comprise medical images of an MRI (magnetic resonance imaging) sequence.

1. A computer-implemented method comprising:
receiving longitudinal medical images of one or more anatomical objects of a patient acquired over a plurality of timepoints;
for each respective timepoint of the plurality of timepoints, extracting features from the longitudinal medical images acquired at the respective timepoint using a machine learning based feature extractor network;
weighting the extracted features for the plurality of timepoints based on a patient atlas of the one or more anatomical objects of the patient;
analyzing the one or more anatomical objects based on the weighted extracted features using a machine learning based task network; and
outputting results of the analysis of the one or more anatomical objects.

2. The computer-implemented method of claim 1, wherein the patient atlas defines a location of the one or more anatomical objects in the longitudinal medical images.

3. The computer-implemented method of claim 1, further comprising generating the patient atlas by:
for each respective timepoint of the plurality of timepoints, generating a segmentation mask of the one or more anatomical objects in the longitudinal medical images acquired at the respective timepoint;
registering the longitudinal medical images for the plurality of timepoints; and
combining the segmentation masks based on the registration to generate the patient atlas.

4. The computer-implemented method of claim 1, wherein the machine learning based feature extractor network is trained by:
receiving training medical images;
degrading the training medical images by applying one or more transformations;
extracting training features from the degraded training medical images using the machine learning based feature extractor network;
reconstructing the training medical images based on the extracted training features using a machine learning based decoder network; and
training the machine learning based feature extractor network and the machine learning based decoder network based on a comparison between the training medical images and reconstructed training medical images.

5. The computer-implemented method of claim 1, wherein weighting the extracted features for the plurality of timepoints based on a patient atlas of the one or more anatomical objects of the patient comprises:
encoding the patient atlas into a patient atlas feature vector; and
combining the patient atlas feature vector and the extracted features as an input token for input into the machine learning based task network.

6. The computer-implemented method of claim 1, wherein the plurality of timepoints comprises a timepoint corresponding to a baseline examination of the one or more anatomical objects, one or more timepoints corresponding to one or more follow-up examinations of the anatomical objects, and a timepoint corresponding to a current examination of the anatomical objects.

7. The computer-implemented method of claim 1, wherein the one or more anatomical objects comprise one or more prostate cancer lesions on a prostate of the patient.

8. The computer-implemented method of claim 7, wherein analyzing the one or more anatomical objects based on the weighted extracted features using a machine learning based task network comprises:
determining at least one of a Gleason grade group score or an indication of progression of the prostate cancer lesions.

9. The computer-implemented method of claim 1, wherein the longitudinal medical images comprise medical images of an MRI (magnetic resonance imaging) sequence.

10. An apparatus comprising:
means for receiving longitudinal medical images of one or more anatomical objects of a patient acquired over a plurality of timepoints;
for each respective timepoint of the plurality of timepoints, means for extracting features from the longitudinal medical images acquired at the respective timepoint using a machine learning based feature extractor network;
means for weighting the extracted features for the plurality of timepoints based on a patient atlas of the one or more anatomical objects of the patient;
means for analyzing the one or more anatomical objects based on the weighted extracted features using a machine learning based task network; and
means for outputting results of the analysis of the one or more anatomical objects.

11. The apparatus of claim 10, wherein the patient atlas defines a location of the one or more anatomical objects in the longitudinal medical images.

12. The apparatus of claim 10, further comprising means for generating the patient atlas by:
for each respective timepoint of the plurality of timepoints, generating a segmentation mask of the one or more anatomical objects in the longitudinal medical images acquired at the respective timepoint;
registering the longitudinal medical images for the plurality of timepoints; and
combining the segmentation masks based on the registration to generate the patient atlas.

13. The apparatus of claim 10, wherein the machine learning based feature extractor network is trained by:
means for receiving training medical images;
means for degrading the training medical images by applying one or more transformations;
means for extracting training features from the degraded training medical images using the machine learning based feature extractor network;
means for reconstructing the training medical images based on the extracted training features using a machine learning based decoder network; and
means for training the machine learning based feature extractor network and the machine learning based decoder network based on a comparison between the training medical images and reconstructed training medical images.

14. The apparatus of claim 10, wherein the means for weighting the extracted features for the plurality of timepoints based on a patient atlas of the one or more anatomical objects of the patient comprises:
means for encoding the patient atlas into a patient atlas feature vector; and
means for combining the patient atlas feature vector and the extracted features as an input token for input into the machine learning based task network.

15. A non-transitory computer-readable storage medium comprising instructions which, when executed by a computer, cause the computer to carry out operations comprising:
receiving longitudinal medical images of one or more anatomical objects of a patient acquired over a plurality of timepoints;
for each respective timepoint of the plurality of timepoints, extracting features from the longitudinal medical images acquired at the respective timepoint using a machine learning based feature extractor network;
weighting the extracted features for the plurality of timepoints based on a patient atlas of the one or more anatomical objects of the patient;
analyzing the one or more anatomical objects based on the weighted extracted features using a machine learning based task network; and
outputting results of the analysis of the one or more anatomical objects.

16. The non-transitory computer-readable storage medium of claim 15, wherein the patient atlas defines a location of the one or more anatomical objects in the longitudinal medical images.

17. The non-transitory computer-readable storage medium of claim 15, wherein the plurality of timepoints comprises a timepoint corresponding to a baseline examination of the one or more anatomical objects, one or more timepoints corresponding to one or more follow-up examinations of the anatomical objects, and a timepoint corresponding to a current examination of the anatomical objects.

18. The non-transitory computer-readable storage medium of claim 15, wherein the one or more anatomical objects comprise one or more prostate cancer lesions on a prostate of the patient.

19. The non-transitory computer-readable storage medium of claim 18, wherein analyzing the one or more anatomical objects based on the weighted extracted features using a machine learning based task network comprises:
determining at least one of a Gleason grade group score or an indication of progression of the prostate cancer lesions.

20. The non-transitory computer-readable storage medium of claim 15, wherein the longitudinal medical images comprise medical images of an MRI (magnetic resonance imaging) sequence.
